(19) United States
(12) Patent Application Publication
(10) Pub. No.: US 2025/0277783 A1
(43) Pub. Date: Sep. 4, 2025

(54) ENGINEERED CANNABINOID BIOMARKER(S), IN VITRO AND IN VIVO TESTING, AND RELATED APPLICATIONS

(71) Applicant: Auxergen Inc., Baltimore, MD (US)

(72) Inventor: Ting-Yu Yeh, Baltimore, MD (US)

(73) Assignee: Auxergen Inc., Baltimore, MD (US)

(21) Appl. No.: 18/594,785

(22) Filed: Mar. 4, 2024

(51) Int. Cl. G01N 33/50; C12N 15/62; G01N 33/68

(52) U.S. Cl. CPC G01N 33/5023; C12N 15/62; G01N 33/68; G01N 2333/4719; G01N 2500/04

(57) ABSTRACT

Methods and applications to label, detect and engineer native or recombinant CB1 and CB2 receptors for cannabinoid chemicals are provided. Also disclosed are uses to detect the dynamics and real time pharmacokinetics of cannabinoid receptors, and purification of cannabinoid receptors in vivo (humans, animals etc.) and in vitro (in the cells).

### FIELD OF THE DISCLOSURE

This invention relates to methods and applications to label, detect and engineer native or recombinant receptors for cannabinoid and hemp chemicals. This invention further relates to applications that utilize the dynamics and pharmacokinetics of cannabinoid receptors, as well as purification of cannabinoid receptors in vivo (humans, animals etc.) and in vitro (in the cells).

### BACKGROUND OF THE DISCLOSURE

An animal model is a living, non-human animal used during the research and investigation of human disease, for the purpose of better understanding the disease process without the added risk of harming an actual human. Testing using animal models are the current and most commonly used conventional methods for testing the efficacy of drugs, including Cannabis or Hemp based drugs. The animal chosen will usually meet a determined taxonomic equivalency to humans, so as to react to diseases or their treatment in a way that resembles human physiology as needed. Many drugs, treatments and cures for human diseases have been developed with the use of animal models. However, there are several downsides to the use of animal models for testing and research of new and existing drugs, some of which include: the death of thousands of animals due to testing; the associated time to collect and analyze data over a large animal population; the inherent error/difficulty in predicting human response based on animal data; and the added expense of R&D that is passed on to the patient/consumer. The use of animal models is expensive, labor intensive, time consuming, involves variability in animals (e.g., mouse populations), is less reproducible, only provides ˜70% accuracy, utilizes secondary effects/signaling, and provides only indirect readout of a drug's function. Thus, new and improved methods are needed and desirable to overcome these deficiencies and increase the efficiency of the drug development process.

### BRIEF SUMMARY OF THE DISCLOSURE

Two receptors (CBI and CB2) are G-protein coupling receptors for cannabis-related (cannabinoid) compounds. Commercially available methods to detect CB1 and CB2 receptor activity are limited to the indirect, downstream (beta-arrestin signaling) assays (Tango GPCR assays, Transfluor Assay, LinkLight, PathHunter etc.). The readout may not be physiological relevant and cannot distinguish different pathways of other GPCRs or any non-specific signal crosstalk. So far there is no specific artificial biomarker in the market to label CB1 and CB2 receptor dynamics and activity in the real time in vitro and in vivo. In this patent, an engineered biomarker for Cannabis is provided. It is designed to be capable of binding with fluorescent molecules that “tag” two Cannabis receptors (CBI and CB2) within a subject (e.g., human cells). This engineered biologic invention enables in-vitro visualization (mapping and quantification) of receptor activity without changing the natural behavior of CB-receptor cells in real-time. One critical difference over conventional Cannabis drug testing methods is that the biomarker enables testing by extracting blood of a subject, injecting the biomarker and observing its impact over time. This can be done directly on human/subject cells rather than requiring hundreds, if not thousands of animal model subjects.

Further, when a Cannabis molecule (e.g., a Cannabis-based drug) is exposed to those same receptors, the biomarker tagging capability of the present invention facilitates real-time observation of the response of the receptors to a drug. Such observation or testing is achieved with 99% accuracy, and testing readout can occur within seconds, rather than hours, relative to conventional animal model testing currently. As a result, the biomarker technology disclosed herein provides higher accuracy, faster throughput, and lower cost, compared to that of animal model-based testing, which utilizes indirect observation of the activity of the receptors using beta-arrestin signaling with only 70% accuracy.

Embodiments of the invention included herein include the engineered biomarker itself; testing methodologies and applications utilizing the biomarker; and related Cannabis and Hemp applications including the following:
Applications and methods designed to specifically label cannabinoid receptors with artificially designed peptides (SEQ ID NO: 1 and 2);
Use of the biomarkers for real-time detection of cannabinoid receptors in living cells;
Use of the biomarkers for measurement of pharmacokinetics of cannabinoid receptors after drug treatment;
Use of the biomarkers for purification of cannabinoid receptors in vitro and in vivo;
Use of the biomarkers to label cannabinoid receptors in animal models (e.g., mice etc.);
Use of the biomarkers for testing related to suppressing immune system response;
Use of the biomarkers in testing, labeling, and determining the safety and efficacy of their current and future cannabinoid-based drug offerings;
Use of the biomarkers for individual patient testing/personal medicine;
Use of the biomarkers to create taxonomy to describe specific benefits and treatment applications;
Use of the biomarker for testing related to a second pathway (MHC-1 endocytic payhway), for the CB2-receptor that delivers cannabinoids to the immune system;
Design, modification and biological source material related to Cannabis agriculture and cultivation;
Hemp and Cannabis-based anti-inflammatory response studies;
Pain relief and anti-epilepsy properties of CBD, THC, and their derivatives;
Liver protection studies;
Cell death and anti-cancer studies;
Stem cell response and differentiation in the presence of CBD or THC; and
Biomarker and/or Cannabis-based biopesticides and reclamation.

Some of the benefits and advantages of these technologies include: real-time observation of the response of the receptors to the drug; higher accuracy, faster throughput, real-time and direct readout, lower cost, faster testing various cell types; more accurate, not subject to artificial signaling crosstalk, and enables more accurate determinations of specific descriptions of the nature of a drug's response, and ultimately higher quality dosing and toxicity determinations etc. for patients.

The embodiments provided herein represent a breakthrough enabling technology for the study of the pharmacokinetics (PK) of cannabinoids. It will enable drug-designers to rapidly screen individual cannabinoids and cannabinoid combinations for desired characteristics by demonstrating their actual in-cell behavior in-vitro. And, it does so faster and with no signal interference experienced by current cannabis PK assays.

Lastly, as a result of the biomarker provided herein and related testing, Cannabis related products can be made cheaper and potentially more potent or effective because pharma companies are able to more accurately determine toxicity, dosage/concentration, duration (e.g., some drugs impact the binding to a receptor by changing the angle of receptor) of a given drug.

In view of these benefits and advantages and others summarized herein, embodiments of the biomarker technology may be of great value to pharma companies looking to evaluate the efficacy of Cannabis-based drugs. In addition, because the biomarker technology can provide a faster and lower cost alternative for testing various cell types, pharma companies will also have the ability to make more accurate and specific claims about the nature of their drug's response, ultimately resulting in higher quality dosing and toxicity determinations, etc. for patients.

### BRIEF DESCRIPTION OF THE DRAWINGS

The present disclosure is illustrated and described herein with reference to the various drawings, in which like reference numbers are used to denote like system components and/or method steps, as appropriate, and in which:

FIG. 1A illustrates two methods of labeling cannabinoid receptors with AxCBMl and AxCBM2 biomarkers;

FIG. 1B is the column-purified AxCBMl and AxCBM2 biomarkers.

FIG. 1C is the results of the direct ligand-receptor assay of AxCBMl and AxCBM2 biomarkers.

FIG. 1D is a summary of the advantages of AxCBMl and AxCBM2 biomarkers compared to other commercial assays;

FIG. 2A-2C are three examples of real time visualization of AxCBMl and AxCBM2 biomarkers-labeled CB receptors in the cell;

FIG. 3A-3C illustrate applications of AxCBMl and AxCBM2 biomarkers; and real-time pharmacokinetic curve using a fluorescence microplate reader; and

FIGS. 4A-4C are examples of real-time pharmacokinetic measurement of CB2 receptor after cannabidiol (CBD) treatment using AxCBM2 biomarker;

FIG. 5A illustrates methods and applications of affinity purification of cannabinoid receptors with AxCBMl and AxCBM2 biomarkers;

FIG. 5B is an example of using AxCBMl biomarker to pull down CBl receptor from rat brain lysate;

FIG. 6 is an application of AxCBM biomarkers to label cannabinoid receptors in the animal model;

FIGS. 7 are examples of decay and recycling kinetics of CB receptors m usmg AxCBMl or AxCBM2 biomarker in flow cytometry; and

FIGS. 8A-8B are examples of automatic screening assays in flow cytometry.

FIG. 9 are examples of flow cytometry analysis of the level of surface, internalized, and recycled CB2 receptor using AxCBM2 biomarker and MHC-1 in human immune cell under cannabidiol (CBD) treatment.

The Appendix of Figures includes additional description, embodiments, a glossary and other information relevant to embodiments of Applicant's invention.

### DETAILED DESCRIPTION OF THE DISCLOSURE

The present invention provides an engineered biomarker and applications and methods to specifically label cannabinoid receptors with artificially designed peptides (the “AxCBMl and AxCBM2 biomarkers” or “biomarker”); use of the biomarker for real-time detection of cannabinoid receptors in the living cell; use of the biomarker for measurement of pharmacokinetics of cannabinoid receptors after drug treatment; use of the biomarkers for purification of cannabinoid receptors in vitro and in vivo; and use of the biomarkers to label cannabinoid receptors in the animal models (e.g., mice, other subjects).

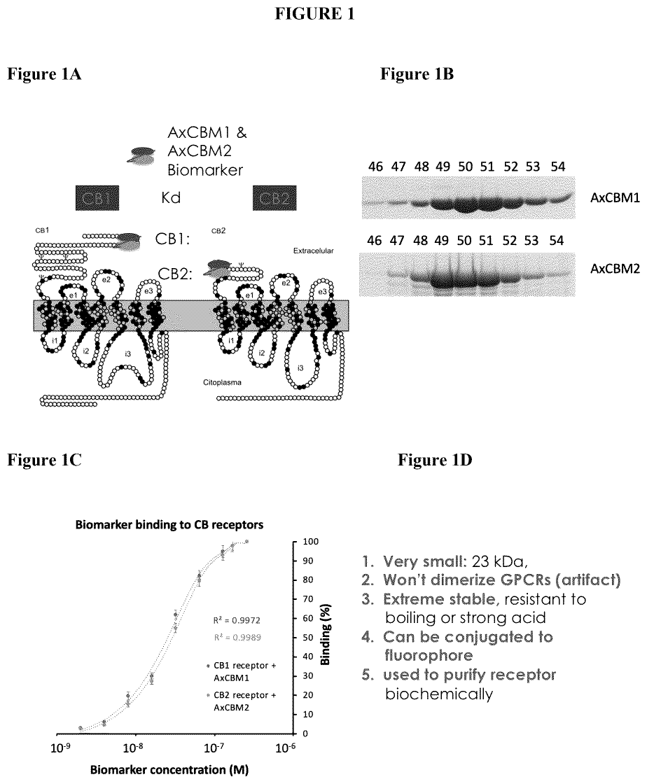

Figure IA illustrates embodiments of methods to label cannabinoid receptors with AxCBMl and AxCBM2 biomarkers according to the present invention. AxCBMl and AxCBM2 biomarkers able to recognize cannabinoid receptor 1 or 2 (hereinafter “CB1 or CB2”) are used to tag these receptors in vitro and in vivo. The affinity (Kd) for AxCBMl and AxCBM2 biomarkers to CBI and CB2 receptors are 13.6 and 5 nM, respectively. Figure lB is Streptactin agarose-purified recombinant AxCBMl (upper) and AxCBM2 (lower) biomarker proteins were chromatographed on a gel filtration column. Individual column fractions (number on each figure) were analyzed by a protein gel and Coomassie Blue staining. Fraction 46-54 of AxCBMl and AxCBM2 biomarkers were pulled together and used for later experiment as the AxCBM1 and AxCBM2 biomarkers. Figure lC shows the binding curves for the binding of AxCBMl (blue) and AxCBM2 (red) to CBl and CB2 receptor, respectively. Estimated dissociation constants (KD) and Hill coefficients of AxCBMl and AxCBM2 biomarker binding to CBl and CB2 receptor are 13.6 and 15.4 nM. FIG. 1D summarizes the advantages of AxCBMl and AxCBM2 biomarkers compared to other commercial assays.

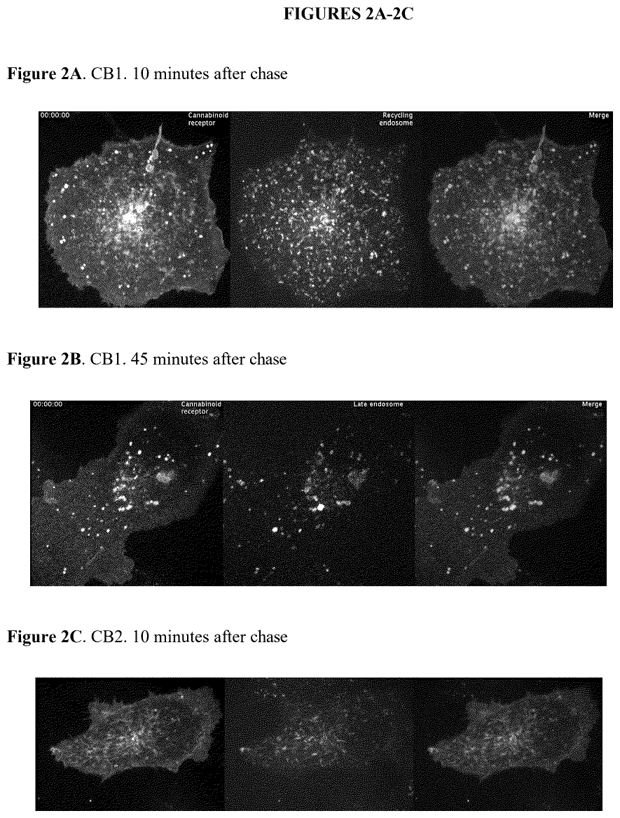

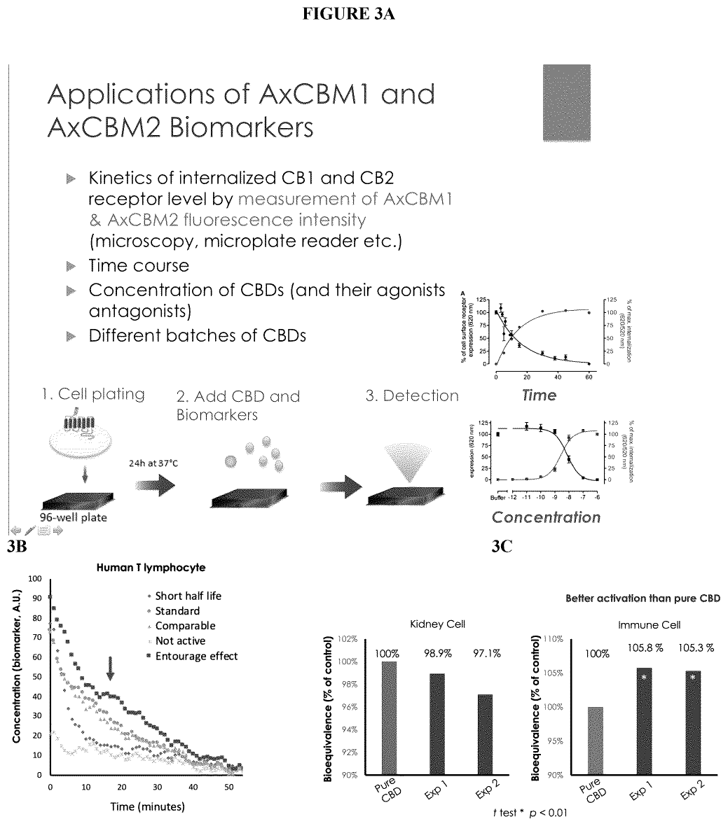

FIGS. 2A-2C provide examples of real time visualization of AxCBMl and AxCBM2 biomarkers-labeled CB receptors in the cell. FIG. 2A is a snapshot of the live-cell movies showing AxCBMl -biomarker-labeled CB1 receptor (green, left) and transferrin-labeled recycling endosome (red, middle) are partially co-localized (right, yellow). It indicates a population of CBl receptor able to recycle back to cell surface within 10 minutes. FIG. 2B is a snapshot of the live-cell movies showing AxCBMl biomarker-labeled CBl receptor (green, left) and LAMPl-labled late endosome (red, middle) are partially co-localized (right, yellow). It indicates that a population of CBl receptor degrades in late endosome after 45 minutes chase (wash out with fresh medium). FIG. 2C is a snapshot of the live-cell movies showing AxCBM2-biomarker-labeled CB2 receptor (green, left) and tubular recycling endosome (red, middle) are co-localized (right, yellow). This result indicates CB2 receptor recycles back to cell surface through a different and faster pathway compared to CBl, which has never been reported in the literature. Our data demonstrate that AxCBMl and AxCBM2 biomarkers can precisely detect cannabinoid receptors in the real time.

FIG. 3A-3C provides applications of AxCBMl and AxCBM2 biomarkers. The cells are plated and cultured in the 96 well plates. Biomarkers, cannabinoids or other medicine combinations (agonists, antagonists, in different concentrations, for different production batches etc.) are loaded on the plate. Direct visualization or measurement of fluorescence intensity of AxCBMl and AxCBM2 biomarkers can be detected under a fluorescence microplate reader. Data can be plotted in a concentration dependent manner or based on the time course to show the drug-response curve (EC50) or half-life of a drug (t1/2). Fluorescent intensity of AxCBM2 biomarkers were detected by a fluorescent microplate reader (Molecular Device) for 60 minutes (FIG. 3B), and the bioequivalence were measured in human kindey (HEK 293T) and immune (Jurkat) cells (FIG. 3C).

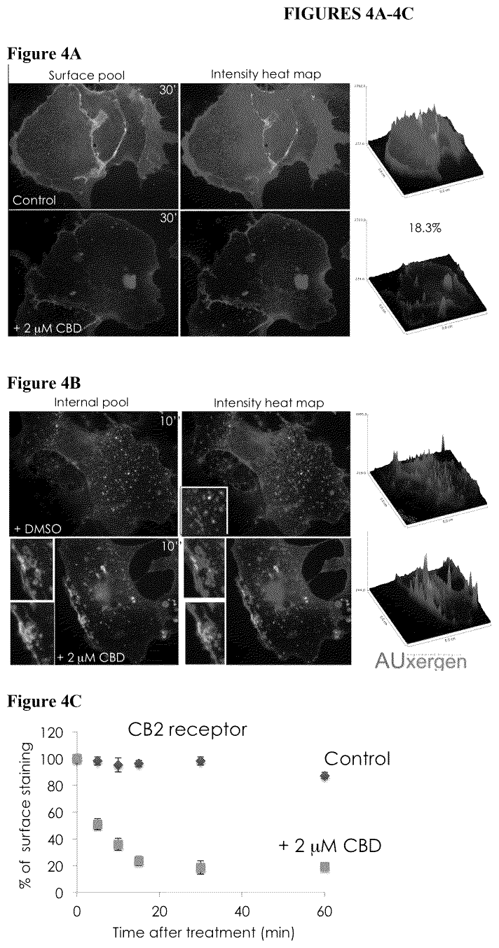

FIGS. 4A-4C provide examples of real-time pharmacokinetic measurement of CB2 receptor after cannabidiol (CBD) treatment using AxCBM2 biomarker. FIGS. 4A illustrates a comparison of surface level of CB2 receptor labeled by AxCBM2 biomarker between control DMSO (upper panel) and 2 μM CBD treatment (lower panel) for 60 minutes. The surface level of CB2receptor was reduced to 18.3% of the control after CBD treatment. FIGS. 4B is a comparison of the internal pool level of CB2 receptor labeled by AxCBM2 biomarker between control DMSO and 2 μM CBD treatment for 60 minutes. It is evident that CB2 receptor highly accumulates in vacuole-like endosome in CED-treated cells. FIGS. 4C is a time course measurement of surface CB2 receptor level in the control and CED-treated cells. The half-life (t1/2) of AxCBM2 biomarker-labeled CB2 receptor internalization is ˜5.3 minutes, which is similar to rat CB2 receptor internalization (t1/2=5.6-15 minutes) measured by several time-consuming and expensive animal model studies (Gonzalez et al., Pharmacol Biochem Behav; 81:300-18.). Our results demonstrated the AxCBM biomarkers establish accurate and quantitative assays to measure the pharmacokinetics of cannabinoid receptors in real time as efficiently as animal studies.

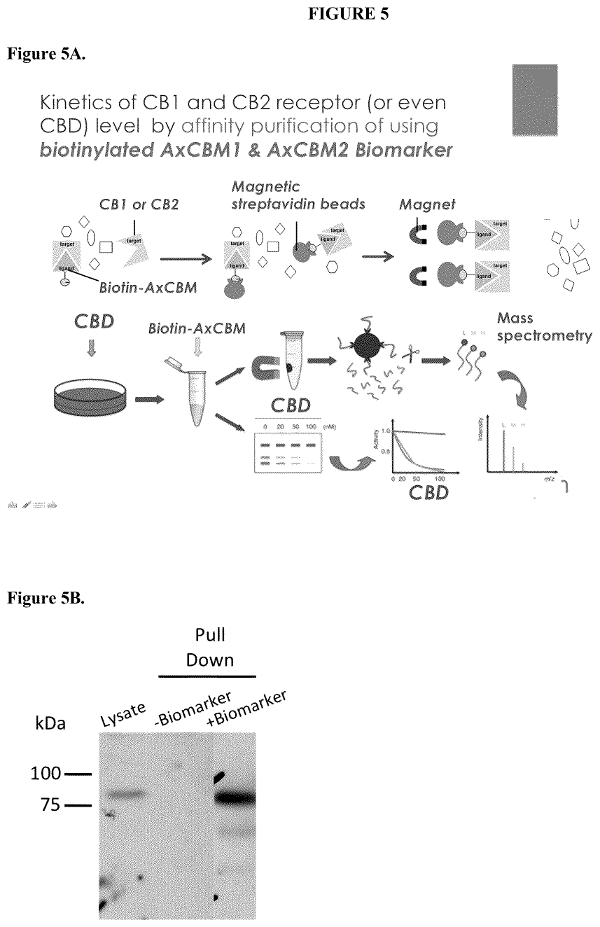

FIG. 5A provides methods and applications of affinity purification of cannabinoid receptors with AxCBMl and AxCBM2 biomarkers. Cells, tissues, or animals are treated with control or cannabinoids (e.g., CBD) in various combinations with other drugs or in different concentrations as requested. After cells or tissues are harvested, protein lysate is prepared and incubated with biotin-conjugated AxCBMl or AxCBM2 biomarkers. CBI or CB2 receptors are pulled down together with AxCBMl or AxCBM2 biomarkers by streptavidin beads. The affinity purified protein samples can be used for further biochemical analysis, such as quantitative immunoblotting or mass spectrometry. FIG. 5B is an example of using AxCBMl biomarker to pull down CB1 receptor from rat brain lysate. Rat brain lysate was incubated with biotin conjugated AxCBMl biomarker and streptavidin beads. The pull-down proteins were analyzed in a 9% SDS polyacrylamide gel and transferred to PVDF membrane. The membrane was immunoblotted with CB1 receptor antibody, which recognizes only one band on the membrane. Our results show that biotin conjugated AxCBMl biomarker can specifically isolate CBI receptor from rat brain lysate. Our biomarkers can provide an accurate biochemical assay to purify and measure the mole number of CB1 and CB2 receptors in a given sample.

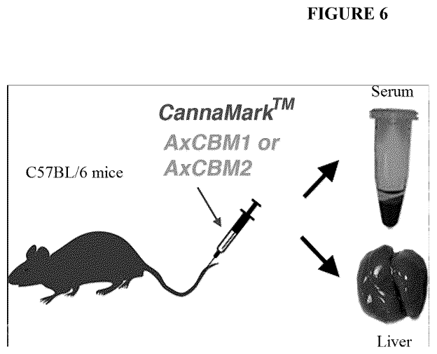

FIG. 6 provides embodiments of the present invention's application of AxCBM biomarkers to label cannabinoid receptors in the animal model. Hydrodynamic injection of AxCBMl or AxCBM2 biomarkers into mice tails was performed according to a known method described in Liu et al. (Gene Therapy 6:1258-66). Eight (8) hours after injection, mice were sacrificed, and their blood and liver samples were collected for further analysis. CB2 receptor signal and localization was shown by AxCBM2 biomarker in the tissue section of mouse liver after injection (green, right). This result demonstrates that our AxCBM biomarkers can also label and measure cannabinoid receptors in regular animal study. Therefore, it can facilitate conventional animal studies by shortening its experimental time with direct measurement.

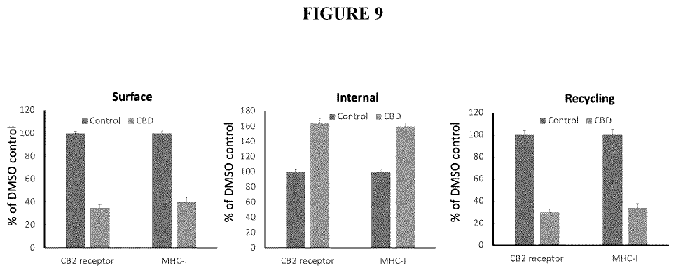

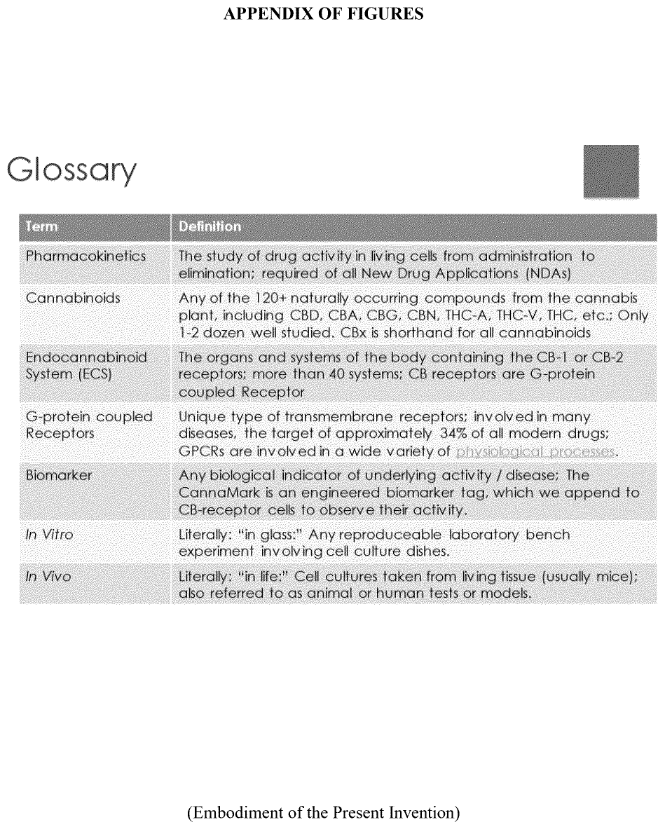

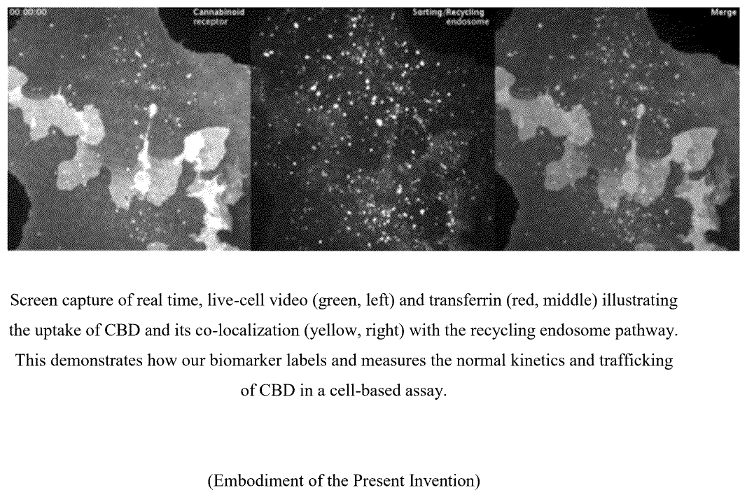

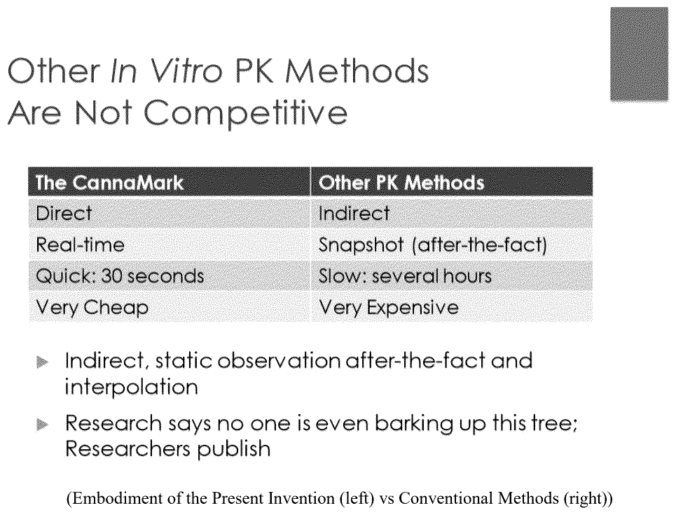

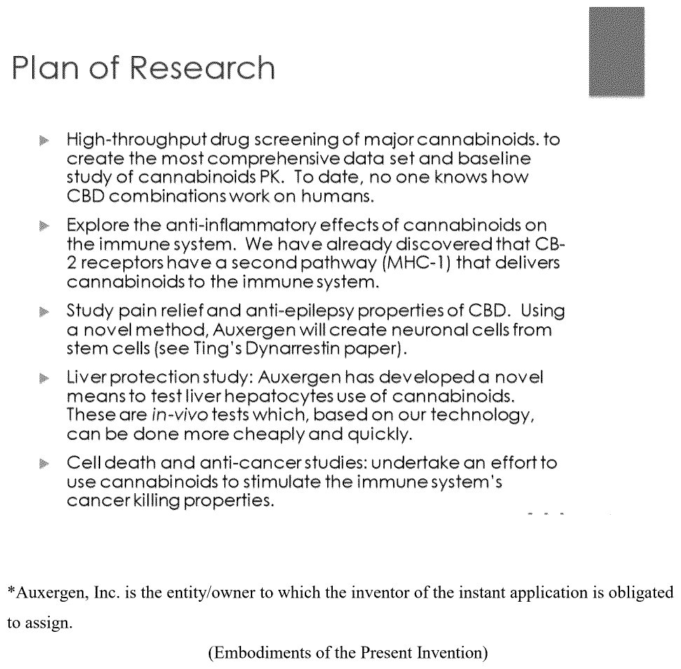

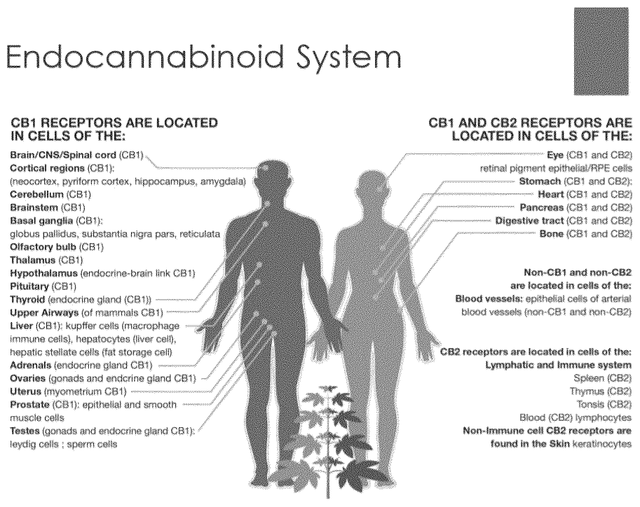

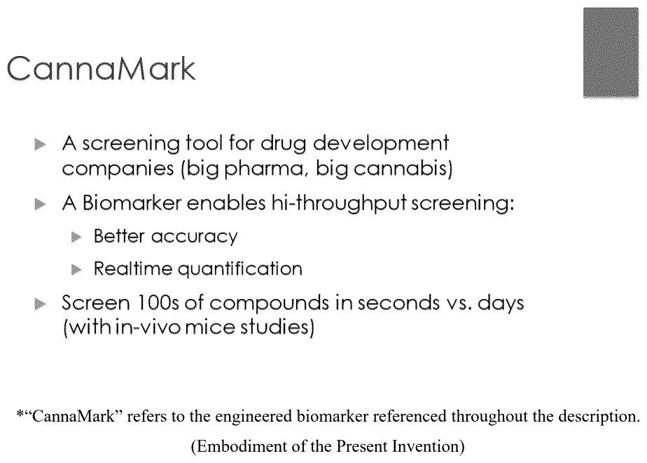

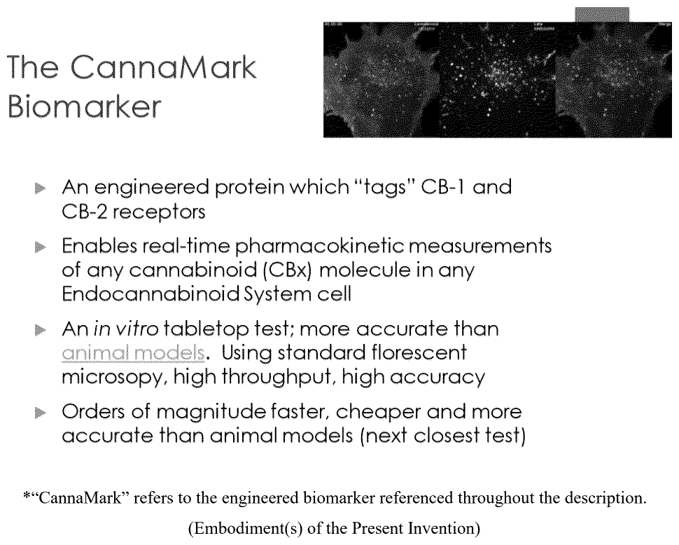

FIG. 9 provides embodiments of the present invention's application of AxCBM2 biomarkers to detect suppresses the surface CB2 and MHC-1 receptor level (left), receptor recycling (right), and receptor accumulation in cytoplasm internally (middle). AxCBM2 biomarker demonstrates 1) that CB2 receptor recycles back to cell surface together with immune complex MHC-1 and 2) we also demonstrated that Cannabidiol (CBD) can suppress MHC-1 and CB2 receptor recycling, providing the first direct evidence how CBD can modulate immune response.

### Exemplary Labeling Methods and Results for Obtaining Concentration Levels of the Full Range of Cannabinoids According to Embodiments of the Present Invention

Adhesion cells with CB receptors were grown on the 96-wells plate overnight to allow attachment. Each treatment condition has three duplicate samples. The following 6 labeling methods are provided as follows:

Method (1). For bacterial expression of AxCBMl and AxCBM2 biomarkers, cells were grown in 250 ml LB culture at 30° C. until O.D. (A600) ˜0.8 and protein expression was induced with 1 mM isopropyl-D-1-thiogalactopyranoside. Cells were shifted to 18° C. and grown overnight. Cells are harvested at 5000 rpm for 30 min in the centrifuge. Cell pellets (from 250 ml overnight culture) were resuspended in 25 ml ice cold lysis buffer A (30 mM Hepes, pH 7.4, 50 mM potassium acetate, 2 mM magnesium acetate, 1 mM EGTA, 10% glycerol, 1 mM dithiothreitol) with protease inhibitor cocktail (Roche complete™ Protease Inhibitor Cocktail Tablets). Cells are sonicated by passage through a French press for 6 times. The cell lysate was clarified by centrifugation at 30,000×g for 15 min at 4° C. The clarified lysate was incubated with 2 ml bed volume DEAE-Sephacel beaed cellulose (Cytiva) for 30 min at 4° C. The bound protein was eluted with NaCl gradient in lysis buffer without KCl. After elution, the concentrated proteins (˜3 mg/ml, 0.5 ml) were passed over a Superose 12 gel filtration column (GE Healthcare) in FPLC to remove aggregates. The peak fractions containing AxCBMl and AxCBM2 proteins were identified by Coomassie Blue staining (FIG. 1B).

The recombinant CB1 and CB2 receptor in carbonate buffer was diluted to a final concentration of 100 ng/μ1. Coat wells of 96-well microtitre plate (ThermoFisher Scientific) with fixed receptor concentration (100 ng/μ1) by pipetting 100 μ1 to each well using a multichannel pipette. Exclude outer walls of the plate to avoid well edge artifact. Cover the plate with a lid and incubate the plate at 4° C. overnight. The coating solution was removed by tilting the plate against the sink and wash the plate 3 times with washing solution (phosphate-buffered saline PBS+0.05% v/v Tween 20). The coated plate was blocked by 200 μ1 of 5% BSA solution to each well using a multichannel pipette and incubate the plate for 2 hours. 100 μ1 of AxCBMl and AxCBM2 biomarkers at different concentrations (8 μg/ml, 4 μg/ml, 2 μg/ml, 1 μg/ml, 0.5 μg/ml, 0.25 μg/ml, 0.125 μg/ml, 0.063 μg/ml, 0.031 μg/ml, 0.0 μg/ml) in PBS were added and incubated for 2 hours at room temperature allowing receptor-ligand interaction. The direct ligand-receptor interaction ELISA was performed usmg TMB substrate solutions (Pierce™ TMB Substrate Kit, ThermoFisher Scientific Cat #34021). The absorbance (optical density, OD) was read directly at 450 nm in a microplate reader. The KD value was estimated by fitting the data to the Hill equation (Figure IC).

Method (2). To measure the pharmacokinetic curve of intracellular level (internal pool) of CB receptor, samples on 96 wells plate are first incubated on ice for 15 min. Icy old medium with Alexa488-conjugated AxCBMl or AxCBM2 biomarkers (1 μg/ml) is loaded on the samples at 4° C. for 30 minutes. After rinsed with 37° C. medium quickly for 3 times, cells were treated with various concentration, duration or combination of cannabinoid drugs at 37° C. Internalized CB receptors, labeled by Alexa488-AxCBM1 or AxCBM2 biomarkers (FIG. 2, green), are partially colocalized with recycling endosome (transferrin, red, FIG. 2A), late endosome (LAMPI, red, FIG. 2B), and tubular recycling endosome (red, FIG. 2C). These results demonstrated that our biomarkers act as precise tracers of CB receptors to monitor the real-time receptor kinetic and trafficking in the cell. After chased with medium, medium with anti-Alexa488 antibody, 5 μg/ml FM4-64FX (Thermo Scientific) and 0.1% sodium azide was added to the sample for 15 minutes at 37° C. to quench the surface CB receptor labeling. Each well was washed with culture medium with 0.1% sodium azide for three times before measurement (e.g. by microscopy, microplate reader, flow cytometry etc.) (See, examples in FIG. 4B).

Method (3). To measure the uptake kinetics of CB receptor, samples are loaded with medium containing Alexa488-conjugated AxCBMl or AxCBM2 biomarkers (1 μg/ml), 200 nM tetramethyl-rhodamine, ethyl ester (TMRE), and various concentration and combination of cannabinoid drugs at 37° C. for measurement (FIG. 3A). 200 μl of Alex488-AxCBM1 or -AxCBM2 biomarker in DMEM was added in the 96 well plates with HEK cell (kidney) and Jukat (immune) cell. Data will be automatically collected by SoftMax Pro Microplate Data Acquisition and Analysis Software (Molecular Devices). Bioequivalence (%) of any given cannabinoid compounds or chemicals was estimated according to USA FDA Definition from 21 CPR § 320.1). (FIG. 3B and 3C). The half-life represents the time period for which the drug concentration decreased by 50%. The duration is 5 times of half-life (<3% left).

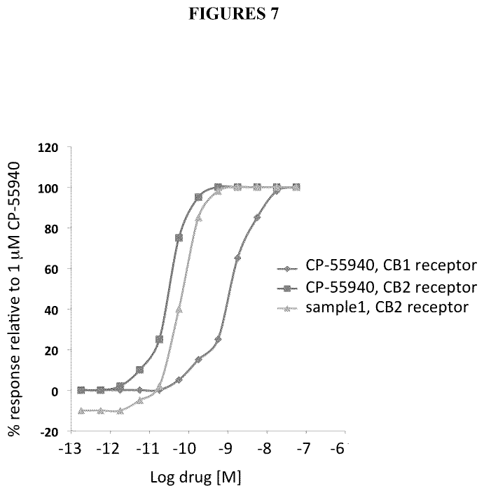

Method (4). To measure the pharmacokinetic curve of surface level of CB receptor, cells were treated with various concentration, duration or combination of cannabinoid drugs at 37° C. 0.1% sodium azide was added to the samples, then each sample was loaded with culture media containing Alexa488-conjugated AxCBMl or AxCBM2 biomarkers (1 μg/ml) and 5 μg/ml FM4-64FX (Thermo Scientific) at 37° C. for 15 minutes. Each well was washed with culture medium with 0.1% sodium azide three times before measurement (e.g. by microscopy, microplate reader, flow cytometry etc.). (Examples in FIG. 4A and 4C). (4) To measure the decay kinetics of CB receptor, samples are loaded with medium containing Alexa568-conjugated AxCBM1 or AxCBM2 biomarkers (1 μg/ml) and various concentration or combination of cannabinoid drugs at 37° C. for 60 minutes. Samples were quickly rinsed with culture medium for 3 times, then immediately loaded with medium containing tryptophan and 1 μg/ml CF680-wheat germ agglutinin (WGA) (Biotium). Samples are incubated at 37° C. for measurement (e.g., by microscopy, microplate reader, flow cytometry etc.). (See examples in FIG. 7A)

Method (5). To measure the recycling kinetics based on intracellular CB receptors, samples are loaded with medium containing Alexa568-conjugated AxCBMl or AxCBM2 biomarkers (1 μg/ml) and various concentration or combination of cannabinoid drugs at 37° C. for 60 minutes. Samples were quickly rinsed with icy cold culture medium for 3 times, then icy cold medium containing tryptophan was loaded on samples and incubated on ice for 15 minutes. Samples were quickly rinsed with 37° C. culture medium for 3 times, then measured in the medium with tryptophan and CF680-WGA at 37° C. (e.g., by microscopy, microplate reader, flow cytometry etc.). (See examples in FIG. 7B).

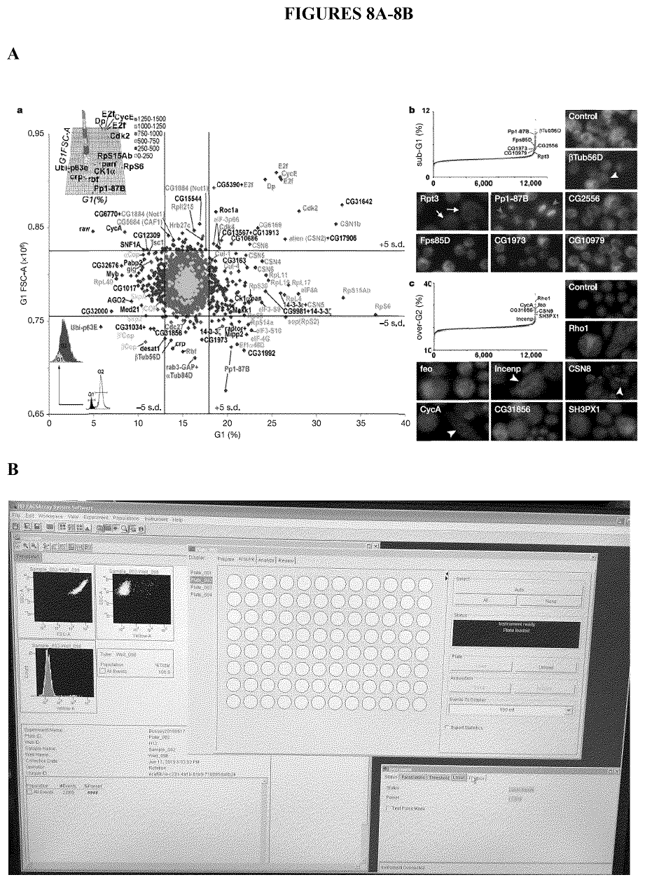

Method (6). Flow cytometry-based recycling kinetics based on the surface CB receptors Cells grown on 10 cm plates were incubated with 10 μg/ml cycloheximide for 1 hour at 37° C. to inhibit protein synthesis. The cells were then treated with various concentration or combination of cannabinoid drugs for 30 minutes at 37° C., to induce CB receptor internalization. Residual surface CB receptor was cleaved by incubation with 0.25% trypsin for 30 mins on ice. Cells were collected by centrifugation and washed in PBS containing 2% PBS (FACS Buffer) prior to resuspension in media containing 10 μg/ml cycloheximide. Cells were incubated at 37° C. to allow CB receptor recycling. Following the appropriate chase time, cells were shifted to ice to prevent further endocytic trafficking of CB receptor and were resuspended to a concentration of l×10⁷ cells/ml in FACS Buffer. To determine the level of CB receptor present on the cell surface, 2×10⁵ cells were aliquoted into triplicate wells of a 96-well plate. Cells were incubated with Alexa488-conjugated AxCBMl or AxCBM2 biomarkers and 5 μg/ml FM4-64FX on ice. Following staining, cells were fixed with 2% formaldehyde in FACS Buffer for 20 mins at room temperature. Residual formaldehyde was removed by washing in FACS Buffer prior to measurement of the mean fluorescent signal for 20,000 events by FACSArray bioanalyzer (BD). Experiments were performed in triplicate and the values were expressed as the level of CB receptor present on the cell surface after each chase time. Data was normalized to the level of CB receptor present on the cell surface at the beginning of medium chase, so that the surface level of CB receptors at time zero was 1. (See examples in FIG. 8B).

Method (7). Data analysis, drug response curve and determination of effective concentration of cannabinoids in the drugs or samples (e.g. foods, drink, lotion, cream, etc.) Standard curve of concentration-dependent drug response is obtained by cannabinoids standards (CBD, CBG, CBN, THC etc., Caymen chemical). The other commercially available standard CP-55940 (Sigma) is a synthetic cannabinoid and acts as a full agonist at CB receptors, with EC50=0.2, 0.3, and 5 nM for CB1, CB2, and GRP55 respectively. Drug responses of test samples with various concentration, treatment duration, or combination of cannabinoid compounds were examined together with 1.0 μM CP-55940. Information of intracellular level, surface level, uptake kinetics, decay kinetics, recycling kinetics of AxCBMl or AxCBM2 biomarker-labeled CB receptors are plotted in a logarithmic scale for drug concentration. Levels of CB receptor activation were calculated and were expressed as percentages relative to the response to 1.0 μM CP-55940. (See examples in FIG. 7C).

To apply AxCBM1 or AxCBM2 biomarker for quantification of the effect of inverse agonist/antagonist co-incubation, measurements of potency (IC50) and efficacy (IMAX) of CP-55940 obtained from the concentration-effect curves were compared between treatments. Antagonist dissociation constants (Kb) were calculated when co-incubation produced surmountable antagonism. When co-incubation resulted in insurmountable antagonism, Kb values were not determined due to the violation of the assumption of competitive antagonism essential for Kb calculation. IC50 and Kb values were then converted to pIC50 and pKb values (plC50 =−Log[IC50] or pKb=−Log[Kb], respectively) so that parametric tests could now be used for statistical comparisons. CP-55940 alone reduced surface AxCBMl or AxCBM2 biomarker-labeled CB receptor levels in a concentration-dependent manner, with a potency (IC50) of 18.7-21.0 nM and efficacy (IMAX) of 36.5-38.6%. In the presence of the competitive CBl receptor mverse agonist/antagonist AM-281 (Sigma), co-incubation resulted in a parallel shift in the concentration-effect curve for CP-55940. AM-281 produced a greater than 8-fold decrease in IC50 of CP-55940 with no change in IMAX (Kb value of 149 nM). Co-incubation with the known competitive CB2 receptor inverse agonist/antagonist AM-630 (Sigma) also leads to a parallel shift in the concentration-effect curve for CP55940, resulting in a Kb value of 57.5 nM.

### Pharmacokinetic Safety Methods and Data

The investigation of cell proliferation by high-throughput analyses of cell populations that contain various proportions of dead or dying and resting or cycling cells is an important tool in drug discovery. FACSArray cytometer and BrdU flow kit to detect S-phase (BrdU Incorporating) cells was used to characterize the nature of proliferating cells. To detect cell death, apoptotic cells are determined by FACSArray Bioanalyzer with annexin V or the vital dye 7-AAD after treated by any sample. Cells were fixed with ice-cold 70% ethanol overnight. The cells were then suspended in PBS containing 30 μg/ml propidium iodide (P1304MP; Molecular Probes) and 30 μg/ml RNase A (RS125; Sigma). After 30 min incubation at 37° C., the cells were analyzed with a FACSArray. Cells that were not immediately analyzed were stored at 4° C., and equilibrated to room temperature before analysis. Alternatively, cells were resuspended in annexin V binding buffer and stained with Alexa Fluor 647 annexin V, followed by PI solution (BD Biosciences). Samples were run in duplicate on a FACSArray flow cytometer, and 10,000 events were captured within the gate each time. Percentages of annexin V-and PI-positive cells were analyzed with FlowJo.

### Automation Methods for Running and Reading Assay Results

Steady-state or real-time measurements of the fluorescence intensity on a SpectraMax Gemini EM plate reader (Molecular Device) were carried out using excitation/emission: 490/520 nm; 544/590 nm, 9-nm bandwidths. PMT sensitivity option was set to high, and 30 reads were taken per each data point. Alexa488 fluorescence intensity in each sample is normalized with fluorescence intensity of FM4-64FX or TMRE. Data was collected by SoftMax Pro Microplate Data Acquisition and Analysis Software (Molecular Devices). Screening was carried out using a benchtop fluorescence plate reader with integrated liquid handling (FlexStation II, Molecular Devices) equipped to perform functional cellular assays and to analyze real time fluorescence kinetic data in the 96-well format. The instrument consists of an incubated cabinet with fluorometer and integrated 96 channel pipettor which is able to transfer compounds from one microplate to the assay plate, allowing rapid kinetic assays. Data acquisition was performed by SoftMax Pro software, and the data were analyzed with Prism (Graphpad) software. Dose-response curves were fitted using Prism (Graphpad) software to the following equation:

$$y = min + ( max - min ) / ( 1 + ( [ Inh \} EC 50 ) n )$$

where y is the relative rate constant for shrinking (max 0=1), [Inh] is the concentration of inhibitor, EC50 is the concentration for 50% inhibition, and n the Hill slope.

Screening of cells response to various concentration, treatment duration, or combination of cannabinoid compounds, or new compounds that may target CB receptors was carried out in FACSArray™ bioanalyzer that features an automated microtiter plate sampler that quickly acquires from 96-well plates (FIG. 8B). Acquisition rates for this instrument are up to 15,000 events per second. High throughput screenings in other non-cannabis applications (such as cell death, bacterial infection etc.) using FACSArray™ bioanalyzer has been previously described (Nature 439:1009-1013; Nature Protocol 11:1531-1553). (See FIG. 8A).

FIG. 7A-7C illustrate examples of decay and recycling kinetics of CB receptors in using AxCBMl or AxCBM2 biomarker in flow cytometry. FIG. 7A shows cell samples were labeled with Alexa568-conjugated AxCBMl biomarker and 2 μM CBD treatment at 37° C. for 60 minutes, followed by medium chase in the presence of tryptophan quencher. Data was collected for 20,000 events by FACSArray bioanalyzer after IO-minute chase. FIG. 7B shows cells were loaded with medium containing Alexa568-AxCBM2 biomarker and 2 μM CBD at 37° C. for 60 minutes. After the surface biomarker was quenched, internal CB2 receptor recycled back to cell surface in the presence of tryptophan. Data was collected for 20,000 events by FACSArray bioanalyzer at IO-minute recycling period. FIG. 7C shows a characterization of Cannabinoid agonist CP 55940 and one cannabinoid sample in decay kinetics assays at the CB1 and CB2 receptor. Our data indicates that AxCBMl or AxCBM2 biomarker will not interfere with CBI receptor decay and recycling back to cell surface. Based on fluorescence intensity of biomarkers, it is feasible to gain the quantification information with a broad linear range (at least for 3 logarithmic scale, 1,000 times difference).

FIG. 8A-8B illustrates examples of automatic screening assays in flow cytometry. FIG. 8A illustrates an example of high throughput screening performed by FACSArray bioanalyzer. (Nature 439:1009-1013). FIG. 8B shows cells in 96 well plates that were treated with various concentration or combination of cannabinoid drugs for 30 minutes at 37° C. Cells were trypsinized, incubated at 37° C. to allow CB receptor recycling, and surface CB receptors were labeled with Alexa488-AxCBMl or AxCBM2 biomarkers. One sample was shown during measurement of the mean fluorescent signal for 20,000 events by FACSArray bioanalyzer. This result shows that AxCBMl or AxCBM2 biomarkers can be applied in a high throughput screening to discover new drugs or formula that target CB receptors.

### Secondary Pathway and Immune Response Application(s)

Jurkat cells were fixed with 2% formaldehyde in PBS for 20 mins at room temperature. Incubate cells with Alexa647-conjugated AxCBM2 biomarkers (1 μg/ml) or 10 μ1/ml Alexa647-anti-MHC I (W6/32) antibody for 30 minutes. The mean fluorescent signal is measured for 20,000 events by FACSArray bioanalyzer (BD). FIG. 9 illustrates examples of use of the biomarker to find/utilize a secondary trafficking and pharmokokinetic pathway for the CB2 receptor (surface, internal, recycling). Because the CB2 receptor can go through a completely different pathway, it can affect how a cell differentiates. The kinetics of CB1 And CB2 are different, for example CB2 kinetics are faster. Because CBD inhibits immune response, embodiments of the present invention include tests that can determine the extent/level to which a particular cannabinoid-based drug can suppress the immune response (FIG. 9).

### Other Application(s)

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes may include, but are not limited to cost, strength, durability, life cycle cost, marketability, appearance, packaging, size, serviceability, weight, manufacturability, ease of assembly, etc. As such, embodiments described as less desirable than other embodiments or prior art implementations with respect to one or more characteristics are not outside the scope of the disclosure and can be desirable for particular applications.

References. Liu et al., Gene Therapy 6:1258-66. Gonzalez et al., Pharmacol Biochem Behav; 81:300-18. Bjorklund, et al., Nature 439:1009-1013; Moor, et al., Nature Protocol 11:1531-1553.

What is claimed is:

1. The cannabinoid receptor biomarkers as provided herein having the amino acid sequences or proteins having the same amino acid sequences where one or more amino acids are added, deleted, or substituted and having receptor activity via binding to cannabinoids.

2. A cannabinoid receptor biomarker protein encoded by a DNA sequence that hybridizes with the DNA sequence or its complementary sequence and having activity to detect cannabinoid receptor activity.

3. The recombinant DNA sequence encoding the protein of any one of claims 1 and 2, wherein said nucleotide sequence consists of SEQ ID NO: 1 and 2 or full length complements thereof; and (b) a nucleotide sequence having at least 80% sequence identity to SEQ ID NO: 1 and 2.

4. A vector comprising the any of the DNA sequences of claim 3.

5. A transformant carrying the vector of claim 4.

6. A method of producing a fusing protein with synthetic amino acid sequences binding to the protein of any one of claims 1 and 2, wherein the method comprises cultivating the transformant of claim 5 (FIG. 1).

7. Methods and applications of screening compounds having ability to trace activity of cannabinoid receptors of any one of claims 1 and 2 (FIGS. 3 and 5), wherein the method comprises:
(a) contacting the proteins of any one of claims 1 and 2 with a substrate (including, but not limiting to, cannabinoids) all to be bound by these proteins in the presence of a test compound to detect the cannabinoid receptor activity of the protein of any one of claims 1 and 2;
(b) comparing the cannabinoid receptor activity detected in step (a) with that detected in the absence of the test compounds and selecting a compound that lowers or increases the activity of cannabinoid receptors of any one of claims 1 and 2; and
(c) purifying the cannabinoid receptor of the protein of any one of claims 1 and 2. (FIG. 5).

8. Methods of screening compounds in live animals having either enhancing or inhibitory activity of cannabinoid receptor activity of the protein of any one of claims 1 and 2 (FIG. 6), wherein the method comprises:
(a) contacting the proteins of any one of claims 1 and 2 with a substrate to be bound by these proteins in the presence of a test compound to detect the cannabinoid receptor activity of the protein of any one of claims 1 and 2; and
(b) comparing the cannabinoid receptor activity detected in step (a) with that detected in the absence of the test compound; and
(c) selecting a compound that lowers or increases the activity of cannabinoid receptors of any one of claims 1 and 2.

9. A composition comprising at least two biomarkers of cannabinoid receptors, or chemical compositions selected from the group consisting of claims 1, 2, 6, 7, and 8.

10. Drug testing, analysis, labeling, or categorization utilizing the cannabinoid receptor biomarkers in any of the above claims or combination(s) thereof.

11. Testing and or analyses of the dynamics and/or pharmacokinetics of one or more of the cannabinoid receptors described in any of the above claims or combination(s) thereof in order to obtain information about the efficacy of a cannabis, or hemp-based drug related to immune response suppression or other diseases (FIG. 9).


SEQUENCE LISTING (2 sequences)

SEQ ID NO: 1 - DNA, 663 bp, synthetic construct
  source 1..663: mol_type = other DNA
tcgtggtggcgtcatgcggcggtatatgaatgggatggtggtggtggtggtggttattcgggttcgtggtggccgccgacgtataataatgaagtaccgctaggtggtggtggtggtggtcatacgtggtggtataatccgccgtcgtatatgggtctagaagcgtcgggtggtggtggtggtggttcgtggtggcgtcatgcggcggtatatgaatgggatggtggtggtggtggtggttattcgggttcgtggtggccgccgacgtataataatgaagtaccgctaggtggtggtggtggtggtcatacgtggtggtataatccgccgtcgtatatgggtctagaagcgtcgggtggtggtggtggtggttcgtggtggcgtcatgcggcggtatatgaatgggatggtggtggtggtggtggttattcgggttcgtggtggccgccgacgtataataatgaagtaccgctaggtggtggtggtggtggtcatacgtggtggtataatccgccgtcgtatatgggtctagaagcgtcgggtggtggtggtggtggttcgtggtggcgtcatgcggcggtatatgaatgggatggtggtggtggtggtggttattcgggttcgtggtggccgccgacgtataataatgaagtaccgctatag

SEQ ID NO: 2 - DNA, 711 bp, synthetic construct
  source 1..711: mol_type = other DNA
ggtacgtggacgtggtggccgccgacgtatgcgggtatggatcatctaggtggtggtggtggtggtacgctatggggtctatttccgccggtatatgaagattcgtttggtctaggtggtggtggtggtggtccgtggacgtcgtggtggccgccggtatatgaaggttcgacgacgaatggtggtggtggtggtggtggtacgtggacgtggtggccgccgacgtatgcgggtatggatcatctaggtggtggtggtggtggtacgctatggggtctatttccgccggtatatgaagattcgtttggtctaggtggtggtggtggtggtccgtggacgtcgtggtggccgccggtatatgaaggttcgacgacgaatggtggtggtggtggtggtggtacgtggacgtggtggccgccgacgtatgcgggtatggatcatctaggtggtggtggtggtggtacgctatggggtctatttccgccggtatatgaagattcgtttggtctaggtggtggtggtggtggtccgtggacgtcgtggtggccgccggtatatgaaggttcgacgacgaatggtggtggtggtggtggtggtacgtggacgtggtggccgccgacgtatgcgggtatggatcatctaggtggtggtggtggtggtacgctatggggtctatttccgccggtatatgaagattcgtttggtctatag